(19) United States
(12) Patent Application Publication
(10) Pub. No.: US 2013/0230142 A1
(43) Pub. Date: Sep. 5, 2013

(54) X-RAY IMAGING APPARATUS

(71) Applicant: CANON KABUSHIKI KAISHA, Tokyo (JP)

(72) Inventors: Jun Murata, Utsunomiya-shi (JP); Takashi Ogura, Tokyo (JP)

(73) Assignee: CANON KABUSHIKI KAISHA, Tokyo (JP)

(21) Appl. No.: 13/775,550

(22) Filed: Feb. 25, 2013

(30) Foreign Application Priority Data
Mar. 1, 2012 (JP) 2012-045678

(51) Int. Cl. A61B 6/00

(52) U.S. Cl. CPC A61B 6/4405
USPC 378/62

(57) ABSTRACT

Provided is an X-ray imaging apparatus, including: an X-ray generation unit for radiating an X-ray; a control unit for controlling the X-ray generation unit; an X-ray reception unit; a storing unit capable of storing the X-ray reception unit for receiving the X-ray radiated from the X-ray generation unit; and a U-shaped arm unit for holding the X-ray generation unit, the control unit, and the storing unit.

### BACKGROUND OF THE INVENTION

1. Field of the Invention

The present invention relates to an X-ray imaging apparatus. Specifically, the present invention relates to an X-ray imaging apparatus including an X-ray tube, a storing unit for storing an X-ray reception unit, and a control unit for controlling the X-ray tube, which are supported by the same arm unit.

2. Description of the Related Art

In recent years, an X-ray imaging apparatus for the purpose of medical diagnosis or the like has been used for emergency treatment or medical care at home because of its enhanced portability due to downsizing and weight reduction of an X-ray generator including the X-ray tube.

Japanese Patent Application Laid-Open No. 2011-56170 discloses a technology in which an X-ray tube is used while suspended vertically above a part of a subject to be inspected by a holding fixture of an assembly type. However, the X-ray imaging apparatus disclosed in Japanese Patent Application Laid-Open No. 2011-56170 has the problem that it requires time and effort to assemble and install the holding fixture prior to radiography being performed. In addition, the X-ray tube and the holding fixture are separate members, and hence there is also a problem in that the portability itself is poor.

Japanese Patent Application Laid-Open No. 2010-57546 discloses a structure in which an X-ray tube and the holding fixture are integrated. According to the structure described in Japanese Patent Application Laid-Open No. 2010-57546, an X-ray imaging apparatus can be quickly installed. However, the X-ray reception unit (X-ray detector) is still a separate member. Therefore, in order to arrange the X-ray tube vertically above the part of a body to be inspected it is necessary to turn on an illumination lamp for illuminating a range equivalent to the X-ray radiation range and to adjust the position of the X-ray tube so that the part of the body to be inspected is placed within the radiation field. In this way, the structure described in Japanese Patent Application Laid-Open No. 2010-57546 has the problem in that it is difficult to perform radiography quickly.

### SUMMARY OF THE INVENTION

An X-ray imaging apparatus includes: an X-ray generation unit for radiating an X-ray; a control unit for controlling the X-ray generation unit; an X-ray reception unit; a storing unit capable of storing the X-ray reception unit for receiving the X-ray radiated from the X-ray generation unit; and a U-shaped arm unit formed of a rod-like member, for holding the X-ray generation unit, the control unit, and the storing unit.

Further features of the present invention will become apparent from the following description of exemplary embodiments with reference to the attached drawings. Each of the embodiments of the present invention described below can be implemented solely or as a combination of a plurality of the embodiments or features thereof where necessary or where the combination of elements or features from individual embodiments in a single embodiment is beneficial.

### BRIEF DESCRIPTION OF THE DRAWINGS

FIG. 1 is a cross-sectional view schematically illustrating a structure of a main part of an X-ray imaging apparatus according to a first embodiment of the present invention.

FIG. 2 is a block diagram schematically illustrating a system configuration of the X-ray imaging apparatus according to the first embodiment of the present invention.

FIG. 3 schematically illustrates a relationship among an X-ray generation unit, a storing unit, a control unit, and an arm unit.

FIG. 4 is a plan view of the X-ray imaging apparatus according to the first embodiment of the present invention viewed from the above.

FIG. 5 schematically illustrates the X-ray imaging apparatus according to the first embodiment of the present invention in a state of being installed in a sideways posture.

FIG. 6 is a flowchart illustrating an example of a process of the X-ray imaging apparatus according to the first embodiment of the present invention.

FIG. 7 is a block diagram schematically illustrating a system configuration of an X-ray imaging apparatus according to a second embodiment of the present invention.

FIG. 8 is a flowchart illustrating an example of a process of the X-ray imaging apparatus according to the second embodiment of the present invention.

FIG. 9 is a block diagram schematically illustrating a system configuration of an X-ray imaging apparatus according to a third embodiment of the present invention.

FIG. 10 is a block diagram schematically illustrating a system configuration of an X-ray imaging apparatus according to a fourth embodiment of the present invention.

### DESCRIPTION OF THE EMBODIMENTS

### First Embodiment

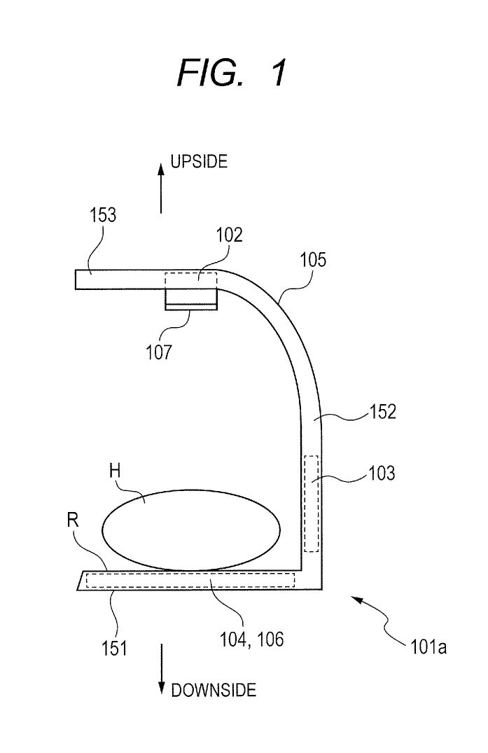

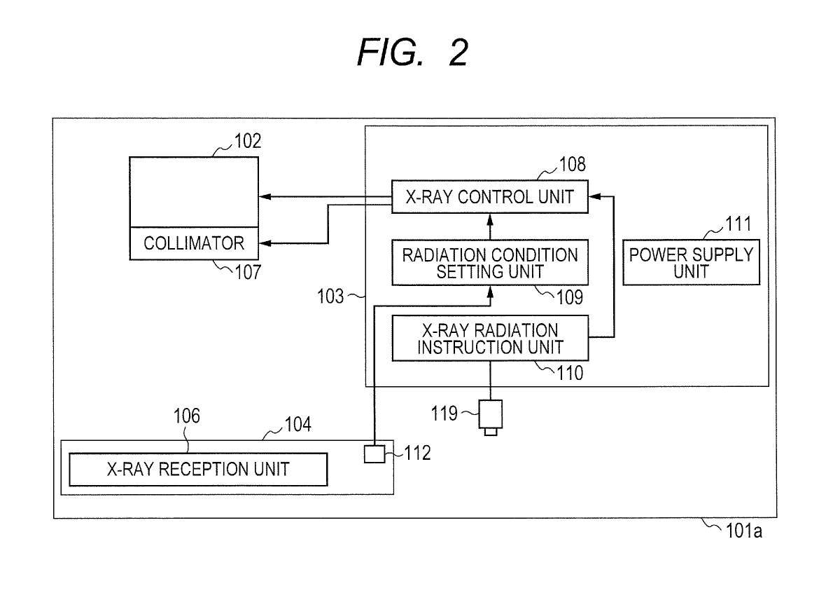

FIG. 1 schematically illustrates a structure of a main part of an X-ray imaging apparatus 101a according to a first embodiment of the present invention. FIG. 2 is a block diagram illustrating a system configuration of the main part of the X-ray imaging apparatus 101a according to the first embodiment of the present invention.

As illustrated in FIGS. 1 and 2, the X-ray imaging apparatus 101a according to the first embodiment includes an X-ray generation unit 102, a control unit 103, a storing unit 104, and an arm unit 105. Further, the X-ray generation unit 102, the control unit 103, and the storing unit 104 are fixed to the arm unit 105.

When the X-ray generation unit 102 receives an X-ray generation signal (described later) from an X-ray control unit 108 of the control unit 103, which is to be described later, the X-ray generation unit 102 generates an X-ray to irradiate a subject H with the X-ray. The X-ray generation unit 102 includes an X-ray tube (not shown) for generating the X-ray, a high voltage generation unit (not shown) for driving the X-ray tube, and a collimator 107 for restricting an X-ray radiation area.

The X-ray tube generates the X-ray, for example, by irradiating an X-ray target made of tungsten or molybdenum with thermoelectrons emitted from a filament heated at high temperature. Further, the X-ray tube and the high voltage generation unit are disposed in the same container. The inside of the container is filled with insulating oil.

The collimator 107 is disposed in a radiation opening of the X-ray generation unit 102. The collimator 107 restricts a radiation range of the X-ray radiated from the X-ray tube and is used for the purpose of reducing generation of scattering rays as much as possible. The collimator 107 is generally movable. Further, an operator can adjust the X-ray radiation range by moving a lead vane disposed on the collimator 107 while confirming the radiation range using a lamp disposed on the collimator 107. In addition, the collimator 107 can also automatically adjust the X-ray radiation range (described later).

The storing unit 104 has a structure of enable storage (in other words, installation) of an X-ray reception unit 106. When the radiography is performed, the X-ray reception unit 106 is stored (installed) in the storing unit 104.

The X-ray reception unit 106 includes a reception surface R and is stored in the storing unit 104 so that the reception surface R faces toward the X-ray generation unit 102. Then, the X-ray reception unit 106 receives, on the reception surface R, the X-ray radiated from the X-ray generation unit 102. As the X-ray reception unit 106, any known unit such as an FPD sensor, a CR cassette, or a film cassette can be used.

The FPD sensor or the like as the X-ray reception unit 106 has a size such as half size, a large quarter size, a quarter size, or the like. The half size has a short side size of 383.5±1.0 mm and a long side size of 459.5±1.0 mm. The large quarter size has a short side size of 307.5±1.0 mm and a long side size of 383.5±1.0 mm. The quarter size has a short side size of 281.5±1.0 mm and a long side size of 332.5±1.0 mm. These sizes are defined by the JIS standard. There are two orientations of the FPD sensor or the like, including a portrait in which the FPD sensor or the like is longitudinal to the subject H and a landscape in which the FPD sensor or the like is transverse to the subject H. One of the orientations is appropriately selected in accordance with a part to be photographed and the purpose of the photography.

In the storing unit 104, any of the FPD sensor, the CR cassette, and the film cassette as the X-ray reception unit 106 can be stored. Further, in the storing unit 104, the size and the orientation of the X-ray reception unit 106 to be stored can are arbitrarily selected. In other words, in the storing unit 104, the FPD sensor or the like in any size can be stored, and the orientation of the FPD sensor or the like to be stored (portrait or landscape) can be arbitrarily selected.

The storing unit 104 is made of a material having light weight and high rigidity such as any synthetic resin material or a carbon fiber reinforced plastic (CFRP). The storing unit 104 has a bag-like structure having an opening formed on one side. Further, the FPD sensor, the CR cassette, or the film cassette as the X-ray reception unit 106 can be inserted into and removed from the storing unit 104 through the opening. The height of the opening is larger than a thickness of 15 mm of the FPD sensor or the like. In addition, the side of the storing unit 104 on which the X-ray enters (the side on which the reception surface R of the X-ray reception unit 106 is disposed) is formed to have a uniform thickness so that the shadow thereof does not fall on the X-ray image.

When the storing unit 104 storing the X-ray reception unit 106 is inserted under the subject H who is lying, the X-ray generation unit 102 can radiate the X-ray toward the subject H.

Inside the storing unit 104, an X-ray reception unit recognizing unit 112 is disposed. The X-ray reception unit recognizing unit 112 recognizes whether or not the X-ray reception unit 106 is stored in the storing unit 104 (installed or not). When the X-ray reception unit 106 is stored, the X-ray reception unit recognizing unit 112 recognizes a type (for example, which one of the FPD sensor, the CR cassette, and the film cassette is used), the size, and the orientation of the X-ray reception unit 106. For instance, identification information is provided on the exterior of the X-ray reception unit 106, and the X-ray reception unit recognizing unit 112 reads this identification information to perform the above-mentioned recognition. As the identification information provided on the exterior of the X-ray reception unit 106, any known one-dimensional code, two-dimensional code, RFID tag, or the like can be used, for example. On the other hand, the X-ray reception unit recognizing unit 112 includes a predetermined reader of various types so as to read for the recognition the identification information provided on the exterior of the X-ray reception unit 106.

Then, the X-ray reception unit recognizing unit 112 generates a recognition signal from a recognition result and sends the generated recognition signal to a radiation condition setting unit 109. The recognition signal contains information regarding whether or not the X-ray reception unit 106 is stored, the type (which one of the FPD sensor, the CR cassette, and the film cassette is used), the size, and the installation orientation of the X-ray reception unit 106.

Note that, when the X-ray reception unit 106 stored in the storing unit 104 is replaced after the X-ray generation unit 102 radiates the X-ray, the X-ray reception unit recognizing unit 112 performs recognition of the X-ray reception unit 106 again and updates the recognition signal. The X-ray reception unit recognizing unit 112 recognizes whether or not the X-ray reception unit 106 is replaced when the recognition signal is updated. When the X-ray reception unit 106 is recognized as the CR cassette or the film cassette (namely, a type that is used through replacement for each photography), and the recognition signal is not updated, the control unit 103 restricts the second X-ray radiation. In other words, when the recognition signal is not updated, the X-ray reception unit recognizing unit 112 determines that the X-ray reception unit 106 is not replaced, and restricts the second X-ray radiation. Thus, double X-ray radiation of the same CR cassette or film cassette can be prevented.

The control unit 103 includes the X-ray control unit 108, the radiation condition setting unit 109, an X-ray radiation instruction unit 110, and a power supply unit 111.

The radiation condition setting unit 109 can set the X-ray radiation condition based on operation by the operator. For instance, a touch panel is disposed on the exterior of the control unit 103, and the operator uses this touch panel to perform the operation of setting the X-ray radiation condition. The X-ray radiation condition includes an X-ray tube voltage, an X-ray tube current, a radiation time, and the like. Note that, the radiation condition setting unit 109 can also set the X-ray radiation condition based on the recognition signal received from the X-ray reception unit recognizing unit 112, and further, the radiation condition setting unit 109 can automatically set the X-ray radiation condition to a recommended value, or a predetermined value. Further, the radiation condition setting unit 109 calculates an X-ray radiation amount or the like based on the set X-ray radiation condition value to generate a radiation condition signal, and sends the generated radiation condition signal to the X-ray control unit 108. The radiation condition signal contains information regarding the X-ray radiation condition such as the X-ray radiation amount.

An operation unit 119 is connected to (or disposed on) the X-ray radiation instruction unit 110. The operation unit 119 includes a button or the like for operating the X-ray radiation instruction unit 110. The X-ray radiation instruction unit 110 sends a signal for instructing the X-ray control unit 108 to radiate the X-ray (hereinafter referred to as an “X-ray radiation instruction signal”) based on the operation of the operation unit 119 by the operator. As the operation unit 119, a deadman's X-ray radiation switch is used. In other words, when the X-ray radiation instruction unit 110 detects that the button disposed on the operation unit 119 is pressed by the operator, the X-ray radiation instruction unit 110 sends the X-ray radiation instruction signal to the X-ray control unit 108. On the other hand, when the X-ray radiation instruction unit 110 detects that the button disposed on the operation unit 119 is released, the X-ray radiation instruction unit 110 promptly sends a signal for instructing the X-ray control unit 108 to stop the X-ray radiation (hereinafter referred to as an “X-ray radiation stop instruction signal”).

Note that, a remote control switch may be used for the X-ray radiation instruction unit 110 and the operation unit 119 so that the operator can perform remote operation. For instance, as the X-ray radiation instruction unit 110 and the operation unit 119, it is possible to use a structure including an infrared reception unit disposed on the exterior of the control unit 103 and a remote switch for transmitting an infrared signal. In this structure, when the X-ray radiation instruction unit 110 detects that the operator has operated the button disposed on the operation unit 119, the X-ray radiation instruction unit 110 sends the X-ray radiation instruction signal to the X-ray control unit 108. On the other hand, when the X-ray radiation instruction unit 110 detects that the operator has released the button, the X-ray radiation instruction unit 110 promptly sends the X-ray radiation stop instruction signal to the X-ray control unit 108. The X-ray radiation instruction unit 110 and the operation unit 119 may have a structure for communicating with the X-ray control unit 108 by a wireless method of the IEEE 802.11 standard, which is widespread as a wireless LAN for personal computers.

When the X-ray control unit 108 receives the radiation condition signal from the radiation condition setting unit 109 and receives the X-ray radiation instruction signal from the X-ray radiation instruction unit 110, the X-ray control unit 108 generates the X-ray generation signal based on the received signals. Then, the X-ray control unit 108 sends the generated X-ray generation signal to the X-ray generation unit 102.

The power supply unit 111 is a power supply for supplying power to each unit of the X-ray imaging apparatus 101a, such as the X-ray generation unit 102. Power supply from the outside to the power supply unit 111 may be commercial power supply of a single phase 100 V, or may be 12 V or 24 V DC power supply from a cigar lighter socket of a car. In addition, the power supply from the outside to the power supply unit 111 may be DC power supply from a battery such as a lithium ion battery, a nickel hydrogen battery, or a fuel cell.

Further, the power supply unit 111 boosts the voltage of the electric power supplied from the outside to approximately 300 V, for example. Then, the power supply unit 111 supplies the electric power with the boosted voltage to the high voltage generation unit disposed on the X-ray generation unit 102.

The control unit 103 is a computer including a central processing unit (CPU), a RAM, a ROM, and the like. The central processing unit executes a computer program so as to cause the X-ray control unit 108, the radiation condition setting unit 109, the X-ray radiation instruction unit 110, and the operation unit 119 to function.

The X-ray imaging apparatus 101a according to the first embodiment of the present invention has a fixed, invariable distance from the X-ray tube of the X-ray generation unit 102 to the reception surface R of the X-ray reception unit 106. Therefore, the collimator 107 can drive the lead vane by a motor or the like based on the recognition signal received from the X-ray reception unit recognizing unit 112, and automatically adjust the radiation range to be a predetermined range. The recognition signal contains information of whether or not the X-ray reception unit 106 is stored, the type (for example, which one of the FPD sensor, the CR cassette, and the film cassette is used), the size, and the installation orientation of the X-ray reception unit 106. The recognition signal generated by the X-ray reception unit recognizing unit 112 is sent to the collimator 107 via the radiation condition setting unit 109 and the X-ray control unit 108 as described later.

Besides, the X-ray imaging apparatus 101a may have a structure further including a display unit (not shown) for displaying the X-ray image received by the FPD sensor when the FPD sensor is used as the X-ray reception unit 106. As this display unit, various types of display apparatus such as a liquid crystal monitor can be used.

The arm unit 105 is a member for fixing the X-ray generation unit 102, the control unit 103, and the storing unit 104. Note that, for convenience sake of description, the “upside” and “downside” of the arm unit 105 are set with reference to the state illustrated in FIG. 1, unless otherwise noted.

The arm unit 105 is formed of a rod-like member. The arm unit 105 includes a portion extending substantially horizontally on the downside (hereinafter referred to as a lower portion 151), a portion extending substantially horizontally on the upside (referred to as an upper portion 153), and a portion connecting the lower portion 151 and the upper portion 153 on the same side (referred to as an intermediate portion 152). Further, the lower portion 151, the intermediate portion 152, and the upper portion 153 are formed integrally. Therefore, the arm unit 105 has a substantially U-shape in side view.

The lower portion 151 and the upper portion 153 of the arm unit 105 are opposed to each other with a predetermined distance therebetween. Further, the subject H can be positioned in the space between the lower portion 151 and the upper portion 153 (for example, an area surrounded by the lower portion 151, the intermediate portion 152, and the upper portion 153, which is hereinafter referred to as an inside).

The storing unit 104 is fixed to the lower portion 151 of the arm unit 105. The control unit 103 is fixed to the intermediate portion 152 of the arm unit 105. The X-ray generation unit 102 is fixed to the upper portion 153 of the arm unit 105. In this way, the storing unit 104, the control unit 103, and the X-ray generation unit 102 are fixed to the arm unit 105. Further, the arm unit 105 supports the X-ray generation unit 102 and the storing unit 104 in a cantilever manner. In other words, end portions of the lower portion 151 and the upper portion 153 on the same side (proximal end portions) are fixed ends connected to the intermediate portion 152. The end portions on the opposite side (distal end portions) are free ends that are not fixed. According to this structure, when the radiography is performed, the X-ray radiated from the X-ray generation unit 102 irradiates the subject H without being blocked.

The X-ray generation unit 102 and the X-ray reception unit 106 are fixed to the arm unit 105 so that a positional relationship therebetween is fixed. Further, only the periphery of the bottom of the storing unit 104 is held in contact with the ground when the X-ray imaging apparatus 101a is installed in such a posture that the storing unit 104 is positioned on the downside as illustrated in FIG. 1. Therefore, even when the subject H lies on a bed or a futon (Japanese bedding), the X-ray imaging apparatus 101a can be used without adjusting the arm unit 105.

The arm unit 105 is made of an aluminum alloy, titanium, a carbon fiber reinforced plastic (CFRP), or the like, for example. According to this structure, weight of the arm unit 105 can be reduced, and rigidity of the arm unit 105 can be enhanced.

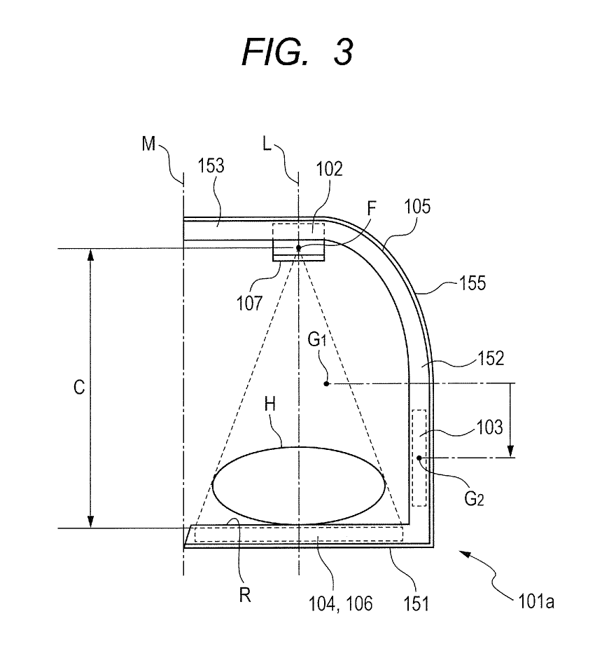

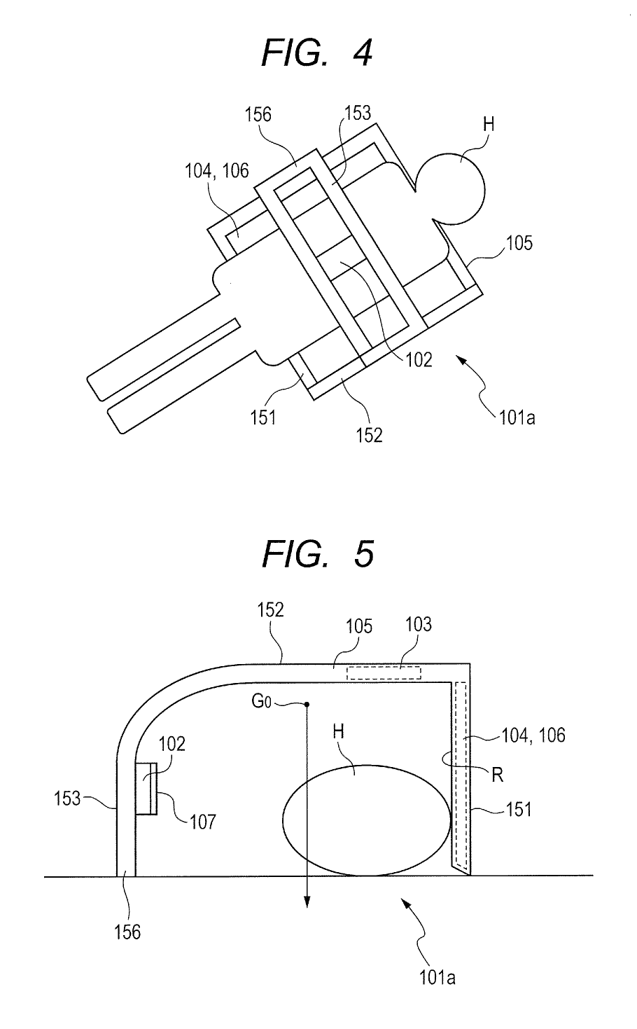

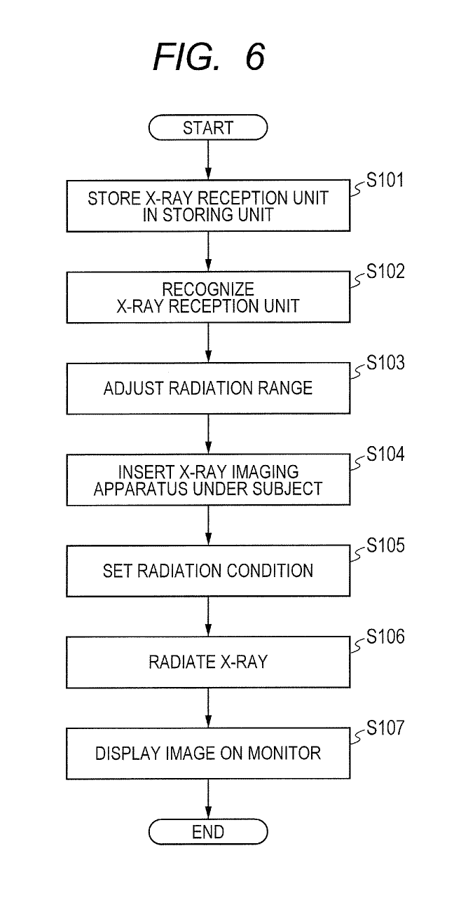

Detailed structure of the arm unit 105 and a relationship among the X-ray generation unit 102, the control unit 103, the storing unit 104, and the arm unit 105 are described with reference to FIGS. 3 to 5. FIG. 3 schematically illustrates the relationship among the X-ray generation unit 102, the control unit 103, the storing unit 104, and the arm unit 105. FIG. 4 is a schematic plan view of the X-ray imaging apparatus 101a in use as viewed from the above. FIG. 5 schematically illustrates the X-ray imaging apparatus 101a in the state of being used in a sideways posture.

As illustrated in FIG. 3, the storing unit 104 storing the X-ray reception unit 106 and the X-ray generation unit 102 are fixed by the arm unit 105 so as to be opposed to each other with a predetermined distance therebetween. The X-ray reception unit 106 is stored in the storing unit 104 in such a posture that the reception surface R faces toward the X-ray generation unit 102. A distance between a focal point F of the X-ray tube of the X-ray generation unit 102 and the reception surface R of the X-ray reception unit 106 stored in the storing unit 104 (distance C in FIG. 3) is preferred to be 1,100 mm or less in view of portability.

A dashed dotted line L in FIG. 3 is the normal passing through the center of the reception surface R of the X-ray reception unit 106. As illustrated in FIG. 3, the X-ray generation unit 102 and the X-ray reception unit 106 are fixed so that the focal point F of the X-ray tube of the X-ray generation unit 102 is positioned above the center of the reception surface R of the X-ray reception unit 106 in the vertical direction (so as to be positioned on the normal L passing through the center of the reception surface R). With this structure, the X-ray radiated from the X-ray tube of the X-ray generation unit 102 passes through the subject H positioned between the upper portion 153 and the lower portion 151 of the arm unit 105 and is received by the X-ray reception unit 106 on the reception surface R. Therefore, a good X-ray image can be obtained.

The control unit 103 is fixed to the intermediate portion 152 of the arm unit 105 and is positioned between the X-ray generation unit 102 and the storing unit 104 in the vertical direction. Further, a center of gravity G₂ of the control unit 103 in the vertical direction is positioned lower (closer to the storing unit 104) than a center of gravity G₁ of the arm unit 105 to which the X-ray generation unit 102 and the storing unit 104 storing the X-ray reception unit 106 are fixed. Therefore, in a state where the X-ray imaging apparatus 101a is installed on a substantially horizontal surface with the lower portion 151 being on the downside, the center of gravity G₂ of the control unit 103 is positioned lower than the center of gravity G₁ of the arm unit 105 to which the X-ray generation unit 102 and the storing unit 104 storing the X-ray reception unit 106 are fixed. With this structure, in a state where the X-ray imaging apparatus 101a is installed in such a posture that the storing unit 104 is positioned on the downside, a position of the center of gravity of the X-ray imaging apparatus 101a can be low. Therefore, even when the X-ray imaging apparatus 101a is placed on an examination table, the posture of the X-ray imaging apparatus 101a can be stabilized enough to prevent falling.

The arm unit 105 further has a function of protecting each of the X-ray generation unit 102, the control unit 103, and the storing unit 104 from an impact applied from the outside (for example, from the side opposite to the space in which the subject H is positioned, and the same is true in the following description).

For instance, as illustrated in FIG. 3, in side view, the X-ray generation unit 102, the control unit 103, and the storing unit 104 are disposed so that the outer circumference surfaces thereof are positioned inside the outer circumference surface of the arm unit 105. In other words, the outer circumference surfaces of the X-ray generation unit 102, the control unit 103, and the storing unit 104 do not protrude from the outer circumference surface of the arm unit 105 in side view. With this structure, it is possible to prevent an impact from being applied from the outside of the X-ray imaging apparatus 101a to the X-ray generation unit 102, the control unit 103, and the storing unit 104, when the X-ray imaging apparatus 101a is touched by a human or an object. In addition, a structure may be adopted, in which a protective member 155 (for example, an external cover member) for protecting the X-ray generation unit 102, the control unit 103, and the storing unit 104 is disposed on the arm unit 105. For instance, a structure can be adopted, in which a rod-like or grid-like member such as a rib or a plate-like member is disposed as the protective member 155 on the outer side of the X-ray generation unit 102, the control unit 103, and the storing unit 104. Even with this structure, it is possible to prevent an impact from being applied from the outside of the X-ray imaging apparatus 101a to the X-ray generation unit 102, the control unit 103, and the storing unit 104 when the X-ray imaging apparatus 101a is touched or struck by a human or an object. Therefore, the X-ray generation unit 102, the control unit 103, and the storing unit 104 can be protected.

The distal end portion of the lower portion 151 of the arm unit 105 (end portion on the opposite side to the intermediate portion 152) is formed into a tapered shape. For instance, the distal end portion of the lower portion 151 of the arm unit 105 is formed into a triangular shape as illustrated in FIG. 3 or into a circular or elliptic shape. According to the structure in which the distal end portion of the lower portion 151 of the arm unit 105 is formed into a tapered shape, the lower portion 151 of the arm unit 105 can be easily inserted under the subject H.

In addition, as illustrated in FIG. 4, a handle 156 to be used for the operator to grip is disposed close to a part of the upper portion 153 of the arm unit 105, in which the X-ray generation unit 102 is disposed. Specifically, as illustrated in FIG. 4, the upper portion 153 of the arm unit 105 has two rod-like members extending substantially horizontally in parallel to each other. Further, the rod-like handle 156 is further disposed to connect the distal end portions of the two rod-like members.

Further, as illustrated in FIG. 5, this handle 156 is formed into such a shape that the posture of the X-ray imaging apparatus 101a is stabilized even when the X-ray imaging apparatus 101a is installed sideways.

FIG. 5 schematically illustrates a state where the X-ray imaging apparatus 101a is installed in the sideways posture. In the sideways posture, the distal end portion of the upper portion 153 or the handle 156 and the distal end portion of the lower portion 151 of the arm unit 105 are held in contact with the ground. In the state illustrated in FIG. 5, the shape and dimensions of the handle 156 are determined so that a center of gravity Gₒ of the X-ray imaging apparatus 101a is positioned between the distal end portion of the upper portion 153 or the handle 156 (a part held in contact with the ground) and the distal end portion of the lower portion 151 (a part held in contact with the ground) of the arm unit 105. For instance, as illustrated in FIG. 3, a straight line M, which passes through the distal end portion of the lower portion 151 and the distal end portion of any one of the upper portion 153 and the handle 156, which has a longer distance from the intermediate portion 152, is set to be perpendicular to the extending direction of the upper portion 153 and the lower portion 151. According to this structure, as illustrated in FIG. 5, the upper portion 153 and the lower portion 151 are substantially perpendicular to the ground surface (for example, the horizontal surface). Further, the X-ray generation unit 102 is fixed to the upper portion 153, the storing unit 104 is fixed to the lower portion 151, and the control unit 103 is fixed to the intermediate portion 152. Therefore, as illustrated in FIG. 5, the center of gravity G₀ of the X-ray imaging apparatus 101a is positioned between the distal end portion of the upper portion 153 or the handle 156 (a part held in contact with the ground) and the distal end portion of the lower portion 151 (a part held in contact with the ground) of the arm unit 105. Therefore, even in the state where the X-ray imaging apparatus 101a is installed in the sideways posture and the distal end portion of the upper portion 153 or the handle 156 and the distal end portion of the lower portion 151 are held in contact with the ground, the posture of the X-ray imaging apparatus 101a does not become unstable.

A gap is formed between the arm unit 105 and the X-ray generation unit 102 as illustrated in FIG. 4. In the same manner, a gap (not shown) is also formed between the arm unit 105 and the control unit 103. Therefore, the operator can grip the arm unit 105 by inserting a hand in this gap. In addition, through these gaps, the operator can confirm the position or the posture of the subject H from the outside of the X-ray imaging apparatus 101a.

Next, an example of a process of the X-ray imaging apparatus 101a according to the first embodiment is described with reference to FIG. 6. FIG. 6 is a flowchart illustrating the example of the process of the X-ray imaging apparatus 101a according to the first embodiment. This process is stored as a computer program (computer software) in the RAM or the ROM of the computer of the control unit 103 except for the process and operation performed by the operator. Then, the central processing unit of the computer of the control unit 103 reads and executes the computer program so that this process is performed.

First, in the first Step S101, the X-ray reception unit 106 is stored in the storing unit 104 by the operator. In Step S102, the X-ray reception unit recognizing unit 112 recognizes whether or not the X-ray reception unit 106 is stored, and further recognizes the type, the size, and the orientation of the X-ray reception unit 106 to generate the recognition signal. Then, the X-ray reception unit recognizing unit 112 sends the generated recognition signal to the radiation condition setting unit 109.

In Step S103, the collimator 107 receives the recognition signal generated by the X-ray reception unit recognizing unit 112 via the radiation condition setting unit 109 and the X-ray control unit 108. Then, the collimator 107 automatically adjusts the X-ray radiation range based on the received recognition signal. If the FPD sensor is used for the X-ray reception unit 106, Steps S102 and 5103 can be omitted by storing the FPD sensor in the storing unit 104 in advance.

In Step S104, the storing unit 104 of the X-ray imaging apparatus 101a (lower portion 151 of the arm unit 105) is inserted under the lying subject H by the operator.

Note that, Step S104 may be performed before Step S101. In other words, a structure may be adopted, in which the storing unit 104 of the X-ray reception unit 106 is inserted after the X-ray imaging apparatus 101a is inserted under the subject H.

In Step S105, the radiation condition setting unit 109 sets the X-ray radiation condition including the X-ray tube voltage, the X-ray tube current, the radiation time, and the like based on operation by the operator using the touch panel or the like disposed on the exterior of the control unit 103. The radiation condition setting unit 109 may set the X-ray radiation condition based on the recognition signal containing information such as the type, the size, and the orientation of the X-ray reception unit 106, which is received from the X-ray reception unit recognizing unit 112 in Step S102. Further, the radiation condition setting unit 109 may also automatically set the X-ray radiation condition to a recommended value or a predetermined value.

In Step S106, the X-ray radiation instruction unit 110 sends the X-ray radiation instruction signal to the X-ray control unit 108 when the deadman's button is pressed by the operator. The X-ray control unit 108 receives the radiation condition signal from the radiation condition setting unit 109 and receives the X-ray radiation instruction signal from the X-ray radiation instruction unit 110. Then, the X-ray control unit 108 sends the X-ray generation signal to the X-ray generation unit 102 at a timing when the X-ray radiation condition is satisfied. When the X-ray generation unit 102 receives the X-ray generation signal, the X-ray generation unit 102 radiates the X-ray.

In Step S107, after the X-ray generation unit 102 radiates the X-ray, the X-ray imaging apparatus 101a displays the photographed image on the display unit (not shown). Thus, the operator can confirm the photographed X-ray image. The method of confirming the photographed X-ray image is different depending on the type of the X-ray reception unit 106 or the like.

As described above, the first embodiment of the present invention provides the structure in which the operator stores the X-ray reception unit 106 in the storing unit 104 in advance in Step S101, and the radiation condition setting unit 109 sets the radiation condition in Step S105. With this structure, the operator can operate the X-ray imaging apparatus 101a to radiate the X-ray only by inserting the storing unit 104 of the X-ray imaging apparatus 101a under the subject H in Step S104 and operating the X-ray radiation instruction unit 110 in Step S106. Further, it is possible to prevent unnecessary radiation or double radiation when the CR cassette or the film cassette is used as the X-ray reception unit 106. Therefore, operability can be improved.

### Second Embodiment

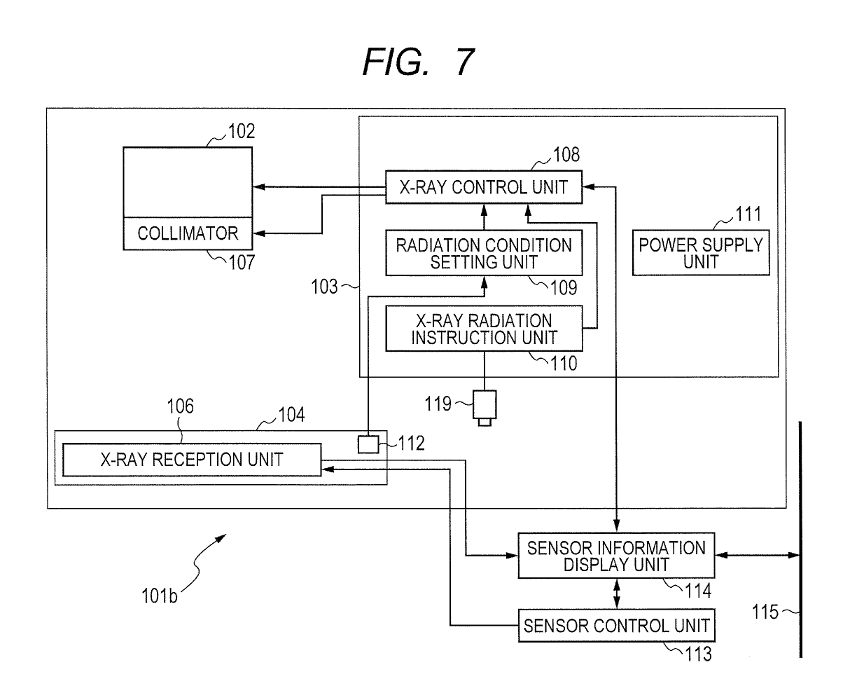

Next, an X-ray imaging apparatus 101b according to a second embodiment of the present invention is described with reference to FIG. 7. Note that, the same reference numerals or symbols are assigned to the same components as those in the first embodiment, and hence redundant description is omitted. FIG. 7 is a block diagram schematically illustrating a system configuration of the X-ray imaging apparatus 101b according to the second embodiment. The X-ray imaging apparatus 101b according to the second embodiment includes an FPD sensor as the X-ray reception unit 106, and is connected to an HIS/RIS server (not shown) or a PACS server (not shown) so as to transmit/receive signals from/to the server(s).

As illustrated in FIG. 7, the X-ray imaging apparatus 101b according to the second embodiment includes the X-ray generation unit 102, the control unit 103, the storing unit 104, a sensor control unit 113, and a sensor information display unit 114. Further, the X-ray imaging apparatus 101b is connected to the HIS/RIS server or the PACS server (not shown) via a network 115 so as to transmit/receive signals from/to the server(s).

The sensor control unit 113 controls the X-ray reception unit 106. The sensor information display unit 114 controls the X-ray control unit 108 and the sensor control unit 113, and also controls a photography sequence of the X-ray imaging apparatus 101b. Further, the sensor information display unit 114 performs display so that the operator can perform setting concerning the radiography. Note that, some operation to perform setting concerning the radiography may be omitted by using information on the subject H, which is transmitted from the HIS/RIS server.

Further, the sensor information display unit 114 displays information on the subject H (patient) (for example, a name, a sex, an age of the patient, a part to be photographed, and the like), and an electronic image, a histogram of the X-ray dose, and the like, which are sent from the FPD sensor as the X-ray reception unit 106. Note that, the sensor control unit 113 and the sensor information display unit 114 are constituted separately from a main body of the X-ray imaging apparatus 101b. In addition, the sensor control unit 113 and the sensor information display unit 114 are constituted integrally, for example. For instance, as the sensor control unit 113 and the sensor information display unit 114, a portable tablet PC which has a plate-like outer shape and includes a touch-panel display and input unit or a portable laptop PC is used.

As communication between the sensor control unit 113 and the X-ray reception unit 106, it is possible to use the wireless communication of the IEEE 802.11 standard, which is used as a wireless LAN for personal computers. In the same manner, as communication between the sensor information display unit 114 and at least one of the X-ray control unit 108, the sensor control unit 113, and the network 115, it is also possible to use the wireless communication of the IEEE 802.11 standard.

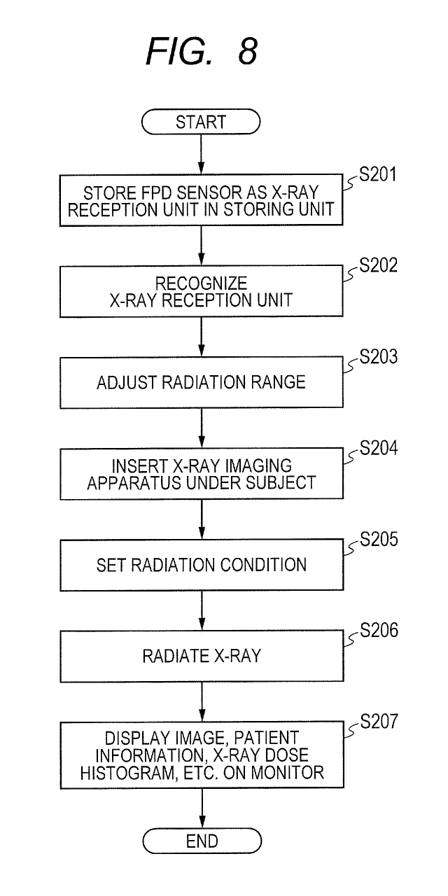

Next, an example of a process of the X-ray imaging apparatus 101b according to the second embodiment of the present invention is described with reference to FIG. 8. FIG. 8 is a flowchart illustrating the example of the process of the X-ray imaging apparatus 101b according to the second embodiment of the present invention.

In the first Step S201, in the same manner as Step S101 of the first embodiment (see FIG. 6), the X-ray reception unit 106 is stored in the storing unit 104 by the operator. In the second embodiment, the FPD sensor is used for the X-ray reception unit 106.

In Step S202, the X-ray reception unit recognizing unit 112 recognizes whether or not the X-ray reception unit 106 is stored, and further recognizes the type, the size, the orientation of the X-ray reception unit 106, and the like to generate the recognition signal. Then, the X-ray reception unit recognizing unit 112 sends the generated recognition signal to the radiation condition setting unit 109. The recognition signal is sent from the X-ray reception unit recognizing unit 112 to the collimator 107 via the radiation condition setting unit 109 and the X-ray control unit 108.

In Step S203, in the same manner as Step S103 of the first embodiment (see FIG. 6), the collimator 107 receives the recognition signal from the X-ray reception unit recognizing unit 112, and automatically adjusts the X-ray radiation range based on the received recognition signal. Note that, Steps S202 and S203 can be omitted by storing the FPD sensor as the X-ray reception unit 106 in the storing unit 104 in advance.

In Step S204, in the same manner as Step S104 of the first embodiment (see FIG. 6), the storing unit 104 of the X-ray imaging apparatus 101b is inserted under the lying subject H by the operator.

Note that, Step S204 may be performed before Step S201. In other words, a structure may be adopted, in which the X-ray reception unit 106 is stored in the storing unit 104 after the storing unit 104 of the X-ray imaging apparatus 101b is inserted under the subject H.

In Step S205, in the same manner as Step S105 of the first embodiment (see FIG. 6), the radiation condition setting unit 109 sets the X-ray radiation condition including the X-ray tube voltage, the X-ray tube current, the radiation time, and the like. The radiation condition setting unit 109 determines a set value based on the operation by the operator using the touch panel or the like disposed on the exterior of the control unit 103. Note that, the radiation condition setting unit 109 may set the X-ray radiation condition based on the recognition signal containing information such as the type, the size, and the orientation of the X-ray reception unit 106, which is received from the X-ray reception unit recognizing unit 112 in Step S202. Further, the radiation condition setting unit 109 may also automatically set the X-ray radiation condition to a recommended value or a predetermined value. Besides, the radiation condition setting unit 109 may set the X-ray radiation condition based on an input from the sensor information display unit 114. Note that, the radiation condition setting unit 109 can omit a part of the input from the sensor information display unit 114 by using the patient information transmitted from the HIS/RIS server. For instance, it is possible to omit an input of the X-ray radiation condition including the X-ray tube voltage, the X-ray tube current, the radiation time, and the like.

In Step S206, in the same manner as Step S106 of the first embodiment (see FIG. 6), the X-ray radiation instruction unit 110 sends the X-ray radiation instruction signal to the X-ray control unit 108 when the deadman's button is pressed by the operator. The X-ray control unit 108 receives the radiation condition signal from the radiation condition setting unit 109 and receives the X-ray radiation instruction signal from the X-ray radiation instruction unit 110. Then, the X-ray control unit 108 sends the X-ray generation signal to the X-ray generation unit 102 at the timing when the X-ray radiation condition is satisfied. When the X-ray generation unit 102 receives the X-ray generation signal, the X-ray generation unit 102 radiates the X-ray.

In Step S207, the X-ray reception unit 106 performs A/D conversion of a charge corresponding to the X-ray dose after passing through the subject H to generate the electronic image, and sends the electronic image to the sensor information display unit 114. The sensor information display unit 114 displays the electronic image together with the patient information, the histogram of the X-ray dose, and the like.

The sensor information display unit 114 performs predetermined image processing on the electronic image in accordance with the operation by the operator. Further, the sensor information display unit 114 performs the process of storing the electronic image in the PACS server.

Note that, when it is desired to repeat the process of the X-ray imaging apparatus 101b along with the changes of the subject H, a part to be photographed, or the like, the process can be performed from Step S204.

As described above, the second embodiment provides the structure in which the FPD sensor as the X-ray reception unit 106 is stored in the storing unit 104, and the information such as the patient information is effectively used from the HIS/RIS server via the network 115. Thus, the operator can omit setting of the X-ray radiation condition including the X-ray tube voltage, the X-ray tube current, and the radiation time. Further, the X-ray imaging apparatus 101b uses the information such as the patient information transmitted from the HIS/RIS server. Therefore, the operator can omit input or setting of the patient information. In addition, because the FPD sensor is used as the X-ray reception unit 106, when repeating the process in the X-ray imaging apparatus 101b along with the changes of the subject H, the part to be photographed, or the like, it is possible to perform the process from Step S204. In this way, compared with the first embodiment, the operability can be further improved.

### Third Embodiment

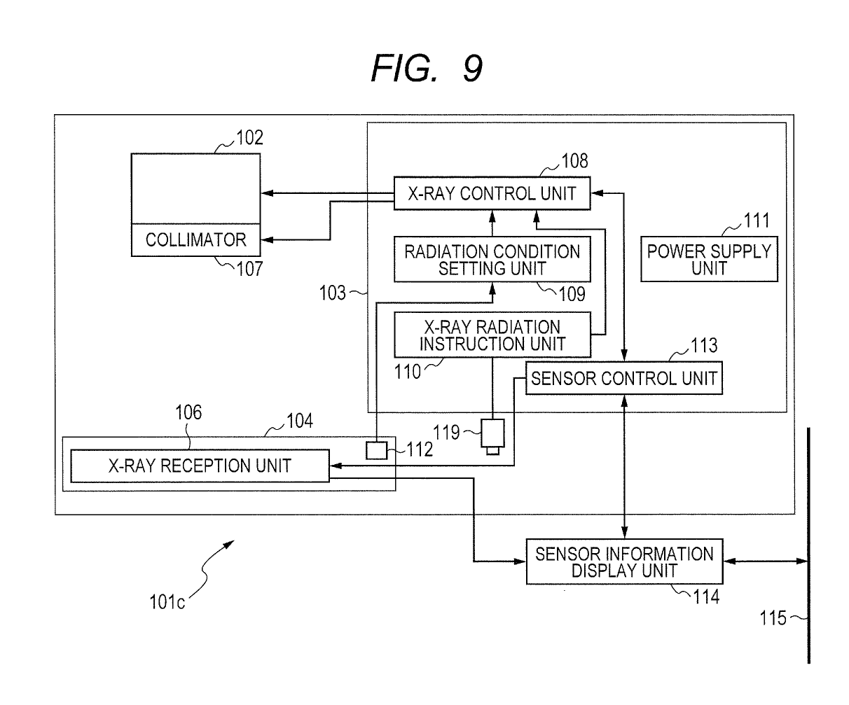

Next, an X-ray imaging apparatus 101c according to a third embodiment of the present invention is described with reference to FIG. 9. FIG. 9 is a block diagram schematically illustrating a structure of a main part of the X-ray imaging apparatus 101c according to the third embodiment of the present invention. Note that, the same reference numerals or symbols are assigned to the same components as those in the first embodiment and the second embodiment, and hence redundant description is omitted.

As illustrated in FIG. 9, the X-ray imaging apparatus 101c according to the third embodiment is different from the X-ray imaging apparatus 101b according to the second embodiment in that the sensor control unit 113 is disposed in the control unit 103. Except for this, the structure is the same as that of the X-ray imaging apparatus 101b according to the second embodiment.

The sensor control unit 113 is disposed in the control unit 103 and is controlled by the control unit 103. With this structure, the sensor information display unit 114 is not required to control the sensor control unit 113. Therefore, the sensor information display unit 114 is not required to have high processing performance. Therefore, as the sensor information display unit 114, for example, a personal mobile phone having a general-purpose OS installed therein can be used.

### Fourth Embodiment

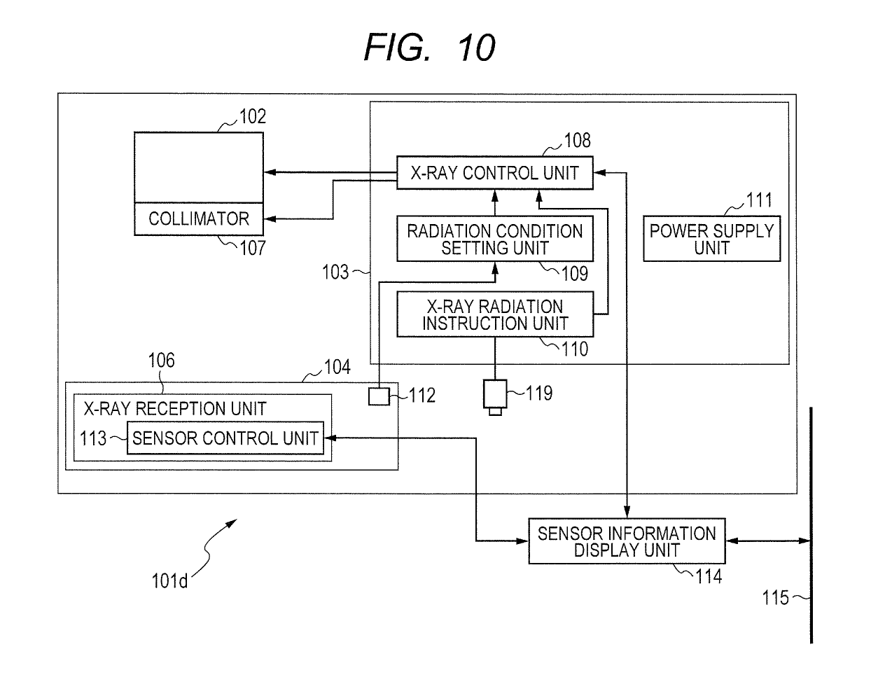

Next, an X-ray imaging apparatus 101d according to a fourth embodiment of the present invention is described with reference to FIG. 10. FIG. 10 is a block diagram schematically illustrating a system configuration of the X-ray imaging apparatus 101d according to the fourth embodiment. Note that, the same reference numerals or symbols are assigned to the same components as those in the first embodiment to the third embodiment, and hence redundant description is omitted.

As illustrated in FIG. 10, the X-ray imaging apparatus 101d according to the fourth embodiment of the present invention is different from the X-ray imaging apparatus 101c according to the third embodiment in that the sensor control unit 113 is disposed in the X-ray reception unit 106. Except for this, the same structure as that in the third embodiment can be used. If the sensor control unit 113 is disposed in the X-ray reception unit 106, all the FPD sensor, the CR cassette, and the film cassette can be stored in the storing unit 104 without disposing an independent sensor control unit 113. Further, with this structure, operability can be improved as in the third embodiment.

As described above, according to each embodiment of the present invention, the X-ray tube, the storing unit for storing the X-ray reception unit, and the control unit for controlling the X-ray tube are fixed to the same arm unit, and hence portability of the X-ray imaging apparatus can be improved. In addition, according to each embodiment of the present invention, it is not necessary to adjust the radiation field position by the X-ray source and to set the radiation condition. Therefore, easiness of installation and operability can be improved.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-045678, filed Mar. 1, 2012, which is hereby incorporated by reference herein in its entirety.

What is claimed is:

1. An X-ray imaging apparatus, comprising:
an X-ray generation unit arranged to radiate an X-ray;
a control unit configured to control the X-ray generation unit;
an X-ray reception unit;
a storing unit arranged to store the X-ray reception unit which is aligned to receive the X-ray radiated from the X-ray generation unit; and
a U-shaped arm unit arranged to hold the X-ray generation unit, the control unit, and the storing unit.

2. An X-ray imaging apparatus according to claim 1, wherein the arm unit is arranged to hold the X-ray generation unit and the storing unit so that a focal point of the X-ray generated by the X-ray generation unit is positioned above a center of a reception surface of the X-ray reception unit stored in the storing unit.

3. An X-ray imaging apparatus according to claim 1, wherein the arm unit is arranged to hold the X-ray generation unit, the control unit, and the storing unit so that a center of gravity of the control unit is positioned closer to the storing unit than a center of gravity of the X-ray imaging apparatus in a state where the X-ray reception unit is stored in the storing unit.

4. An X-ray imaging apparatus according to claim 1, wherein the arm unit is formed of a rod-like member.

5. An X-ray imaging apparatus according to claim 4, wherein the arm unit is arranged to cover an outer side of each of the X-ray generation unit, the control unit, and the storing unit.

6. An X-ray imaging apparatus according to claim 1, wherein:
the arm unit comprises a handle disposed at a distal end portion of a part of the arm unit adjacent to the X-ray generation unit; and
a center of gravity of the X-ray imaging apparatus is positioned between a distal end portion of the storing unit and one of the distal end portion of the part of the arm unit, to which the X-ray generation unit is fixed, and the handle under a state where the distal end portion of the storing unit and the one of the distal end portion of the part of the arm unit, to which the X-ray generation unit is fixed, and the handle are held in contact with a substantially horizontal ground surface.

7. An X-ray imaging apparatus according to claim 1, wherein a distal end portion of a part of the arm unit, on which the storing unit is disposed, is formed into a tapered shape.
